(19) United States
(12) Patent Application Publication
(10) Pub. No.: US 2002/0068015 A1
(43) Pub. Date: Jun. 6, 2002

(54) Apparatus and method for control of ultrafiltration in extracorporeal treatment of blood

(76) Inventors: Hans-Dietrich Polaschegg, Koestenberg (AT); Klaus Sodemann, Lahr (DE)

Correspondence Address: Williams & Associates, 1030 Fifteenth Street, N.W., Washington, DC, 20005-1501, US

(21) Appl. No.: 09/961,095

(22) Filed: Sep. 24, 2001

(30) Foreign Application Priority Data
Sep. 26, 2000 (DE) 11862300

(51) Int. Cl. A61M 1/14; A61M 37/00; C02F 1/44; B01D 61/00; A61M 1/36

(52) U.S. Cl. 422/44; 210/650; 604/5.01; 604/6.09; 210/321.71

(57) ABSTRACT

An apparatus and method for alleviation of symptoms of inappropriate blood pressure and cramping from the removal of water during ultrafiltration hemodialysis and hemofiltration treatment. The invention uses oxygen concentration measured by sensors in various ways and at various points in the blood treatment circuitry to estimate parameters necessary to control the rate at which water is removed from the patient's blood.

This Application is substantially a translation of German Patent Application No. 11862300, filed Sep. 26, 2000 and contains no new matter. Applicants therefore claim priority to the German application pursuant to international treaty rights.

### DESCRIPTION OF INVENTION

The current invention comprises methods and apparatus for the control of ultrafiltration during hemodialysis, hemofiltration or hemodiafiltration. These methods are used for the acute and chronic treatment of kidney failure. In a patient with a failing kidney, excess fluid accumulates in the body of the patient. This excess fluid must be removed. First, however, the amount of excess water to be removed must be determined. Second, this water must be removed smoothly in order to avoid side effects such as blood pressure drops and cramps. The first problem, the evaluation of the amount of superfluous water (or alternatively the evaluation of the patient's dry weight) is usually performed by a physician using professional judgment. The difference between the actual weight of the patient and the thus ascertained dry weight equals the amount of excess fluid which must be removed. The second problem, the smooth removal of the superfluous water, can normally be solved by long treatment times. Side effects are rare with treatment times of eight hours three times per week. Alternatively, treating daily but for a shorter period is also possible without inducing side effects.

Long treatment times, however, are not only a burden for the patient but also result in high costs for the health care system. Therefore an early goal of medical providers was to reduce the overall length of treatment by reducing treatments to four to five hours with increased frequency of treatments.

The first step in implementing this change was the development of devices allowing controlled removal of fluid. Such devices are now the state of the art. An example of a device for volumetric fluid control is described by the German patent DE2858205. With such a device fluid can be extracted from the extracorporeal circuit with a constant prescribed rate through the dialyzer or filter membrane.

Due to the fact that the largest part of the extra fluid is stored in the extracellular space of the patient rather than in the blood and that the refilling rate of the fluid cannot be manipulated, fast fluid removal often results in drastic reduction of blood volume followed by symptoms such as blood pressure drops or cramps. It was recognized early on that these symptoms are related to the effective volume of circulating blood. However, no practical method has yet been found for measuring the patient's total blood volume at each treatment despite the fact that methods for measuring changes in blood volume have existed for some time. Sensors which measure hematocrit, hemoglobin, or total protein of blood can be used for this purpose. Physically this can be done by methods such as measuring the optical density, the density, the electric conductivity, or the viscosity of the blood. One such method is described in the German patent application DE 3827553. Another approach consists of a device for measuring hematocrit employing optical sensors described in U.S. Pat. No. 5,499,627. The device described in 5,499,627 uses several wave lengths for elimination of geometric constants and sources of systematic error. One of these sources of systematic error is the oxygen saturation dependence of the optical absorption of hemoglobin. By using such a device not only is measurement of the hematocrit possible but also simultaneous measurement of the oxygen saturation of blood. The company In-Line Diagnostics of Utah produces and markets a device of this type known as the “Crit-Line Monitor.” (By using such oxygen saturation measurement in the extracorporeal circuit, the influence of sleep apnea (breathing cessation) has been clinically studied.)

Until the current invention, no generally applicable rule has been found to prospectively prevent side effects. Attempts have been made to control the ultrafiltration rate as function of the blood volume change. A method for reducing the number of symptoms in clinical trials in approximately 50% of the patients is based on the measurement of the hematocrit at which symptoms, e.g., blood pressure drops occur. This hematocrit is equivalent to a defined, albeit unknown, blood volume at which symptoms occur. During subsequent treatments, ultrafiltration is stopped before this hematocrit value is reached. The hematocrit of the patient is not only influenced by the fluid load but also by the rate of formation of new blood cells. This rate is influenced by the application of erythropoietin, a hormone influencing the production of red blood cells. Because of this influence, the definition of a clinical hematocrit limit is only possible for a limited duration.

Attempts for predicting the onset of symptoms from the time derivative of blood pressure changes have also so far been unsuccessful. The problem is that during dialysis the blood volume changes only a relatively small amount, up to a maximum of 28%, and that it has been found that during the treatment symptoms do not usually occur at the lowest blood volume measured. Although devices for measuring the blood volume changes have been clinically available for several years and such devices have been integrated into thousands of dialysis machines, there has been no amelioration of the side effect situation.

Normally an arteriovenous fistula is used for the extracorporeal blood treatment in patients with kidney failure. For medical reasons, this is no longer possible with an increasing number of patients. These patients are then treated employing central venous catheters with tips placed in the right atrium.

During hemodialysis treatment of patients using central venous catheters as blood access and with the Crit-Line Monitor from In-Line-Diagnostics for monitoring it was recognized surprisingly that the oxygen saturation of the extracorporeal blood dropped dramatically and immediately before the occurrence of symptoms in spite of the fact that blood volume did not similarly fall. Subsequent reflection on this observation resulted in the conclusion that more oxygen is extracted from blood in the right atrium because of the reduction of effective circulating blood volume. Because of mixing induced by the blood pump it is common for venous blood to mix from the upper and lower vena cava. The oxygen saturation measured is also approximately equivalent to the mixed venous saturation. The mixed venous saturation measured on mixed blood from the upper and lower vena cava is a parameter known in cardiology and intensive care medicine that is normally measured with the help of a special catheter in the pulmonary artery (continuous fiber optical method or periodic blood sampling and measurement with an oxymeter).

Mixed venous saturation is a measure for the oxygen consumption of tissue and organs and is characterized by the difference between the amount of oxygen in arterial and venous blood. In healthy humans, mixed venous saturation decreases under physical stress as oxygen demands of the organs increase. In order to compensate, cardiac output is increased (increase of heart rate) to guarantee oxygen supply to muscles and inner organs. Although a patient does not perform physical work during a hemodialysis treatment, the effective circulating blood volume is reduced without an adequate increase of the heart rate when the ultrafiltration rate exceeds a critical limit resulting in the reduction of the venous saturation. This phenomenon occurs because the relative extraction of oxygen from arterial blood increases at constant oxygen demand. The reduction of the oxygen saturation is correlated to the reduction of the cardiac output (the Fick principle). A dramatic drop results in inadequate oxygen provisions to tissue which is possibly a precursor for symptoms as blood pressure drops or cramps.

For healthy persons with arterial oxygen saturation of 92-100%, mixed venous saturation at rest is approximately 70%. That level is also normal for dialysis patients unless there is other organ damage, e.g., progressing heart failure which influences oxygen extraction. It has been shown that symptoms are more frequent when the oxygen saturation of blood extracted through central venous catheters decreases to 30% or below. Avoiding symptoms by monitoring the decrease of the oxygen saturation and reducing the ultrafiltration rate before the 30% limit is possible. It has further been shown that the oxygen saturation of patients with heart insufficiency is below 70% at the beginning of treatment and that the oxygen saturation increases to almost 70% during treatment subsequently followed by a decrease.

It is therefore proposed to control the ultrafiltration unit of a hemodialysis, hemofiltration or hemodiafiltration device by reference to the oxygen saturation in extracorporeal blood. In the simplest version, an oxygen saturation measurement device is equipped with an adjustable alarm limit and an alarm signal that is initiated when the oxygen saturation decreases below the prescribed limit, thereby alerting the operator of the hemodialysis machine to switch off ultrafiltration. In a further improvement, this switching off can be done automatically. A further improvement allows proportional control of the ultrafiltration as function of the deviation of the oxygen saturation from the initial value. Alternatively the rate of change of the oxygen saturation by itself or in combination with any of the above methods can also be used. The control software can be based on known algorithms such as PD control or fuzzy logic.

Control with one of these algorithms can be performed so an initial ultrafiltration rate is adjusted so that it is larger than the rate calculated from ultrafiltration volume and dialysis time and subsequently the ultrafiltration stops or is reduced if the oxygen saturation decreases or the prescribed ultrafiltration volume is achieved.

Preferably blood must be withdrawn through a double lumen catheter with tips positioned in the right atrium. Alternatively, blood can be withdrawn through a single lumen catheter and re infused through another pathway, e.g., through a peripheral shunt (fistula) or a short catheter not leading to a central vein. Through this means it is possible to monitor cardiac output continuously in resting patients. It is therefore possible to validate therapeutical measures (e.g., application of cardio-vascular medication) in cardiac patients not requiring dialysis by means of this method. Alternatively this method can be used to intermittently measure cardiac output by injection of cold saline (thermodilution) and also allows permanent monitoring of a critical haemodynamic parameter without additional staff.

Most of the oxygen in blood is bound to hemoglobin. This oxygen is in equilibrium with physically dissolved oxygen in plasma. This equilibrium is described by the oxygen saturation curve wherein the oxygen saturation of hemoglobin is a function of the oxygen partial pressure in plasma. This curve is approximately linear within the range in which an intervention, e.g., the reduction or stop of ultrafiltration, is done.

Dialyzers not only allow the exchange of dissolved solid substances but also the exchange of dissolved gases. It has been found that the oxygen partial pressure in spent dialysate correlates with the oxygen saturation in blood. The method for controlling ultrafiltration by oxygen saturation can also be performed with the help of oxygen partial pressure sensors in spent dialysate. In hemofiltration, oxygen partial pressure can be measured directly in the filtrate. Because this is done anaerobically, the oxygen partial pressure corresponds directly with the oxygen partial pressure of the plasma as described in German Patent No. DE3616062.

Dialysate for hemodialysis contains an unknown amount of oxygen, depending on the quality of degassing. An additional oxygen sensor can therefore be placed upstream of the dialyzer in the dialysate circuit and the oxygen content of the plasma can be calculated from the partial pressure differential and the dialyzer clearance for oxygen. Calculating the dialyzer clearance for oxygen from the known clearance for urea is known. The mass transfer coefficient for oxygen is calculated from the mass transfer coefficient for urea using the ratio of the tabulated diffusion constant and the clearance is calculated from the known mass transfer coefficient. Instead of using tabulated values for urea clearances, clearances can be measured as, e.g., described by German Patent No.DE3938662.

### DESCRIPTION OF THE PREFERRED EMBODIMENTS

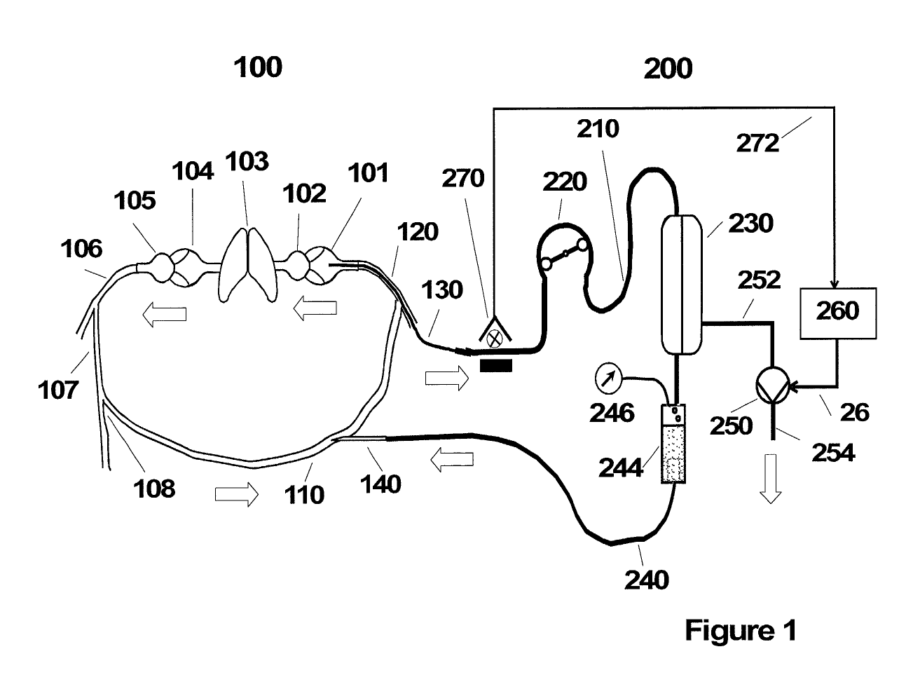

An embodiment of the current invention is shown in FIG. 1. 100 symbolizes the physiological blood circulation and 200 symbolizes the extracorporeal circulation. In the natural circulation blood flows from the right atrium 101 to the right ventricle 102 and is pumped to the lung 103. From there it flows to the left atrium 104 and left ventricle 105 and further to the aorta 106 from where several arteries branch of which only a single one (107) is shown. This artery is, e.g., an artery of the arm connected to a vein of the arm (110) through an anastomosis (108) creating a fistula that can be punctured by a cannula. The vein 110 connects to the central vein (vena cava) 120 leading back to the right atrium 101.

Through the central venous catheter 130 positioned through a peripheral vein into the central vein such that the tip is in the right atrium 101 blood is withdrawn by the blood pump 220. The catheter 130 is connected to the arterial blood tubing system 210 that is connected at the other end to the hemodialyzer, hemofilter, or hemodiafilter 230. Said hemodialyzer, hemofilter, or hemodialfilter is separated into blood and dialysate (or filtrate) part by a membrane not shown. The peristaltic pump is positioned between the catheter and the hemodialyzer. The venous tubing system 240 connects the hemodialyzer 230 with the cannula 140 inserted into the fistula 110. Alternatively the venous tubing system can be connected to a second catheter positioned in an appropriate vein. Also, the venous tubing system can be connected to the venous part of a double lumen catheter. A drip chamber 244, connected to a pressure sensor 246 is integrated into the venous tubing systems. From the filtrate side of the dialyzer/hemofilter/hemodiafilter a conduit 252 branches to the pump 250 allowing controlled removal of fluid by ultrafiltration. The components for conveying dialysate are not shown because they are known to those familiar with the state of the art. The ultrafiltration pump 250 pumps removed fluid through the conduit 254 to the drain. The pump 250 is controlled by the control unit 260 connected to the pump by the electrical line 262.

The optical sensor 270 for measuring oxygen saturation is positioned on the arterial blood tubing system. Said sensor is connected to the ultrafiltration control unit by the signal line 272. The sensor 270 can be positioned in the arterial (210) or, alternatively in the venous (240) part of the blood tubing system because oxygen saturation is only insignificantly altered by hemofiltration or hemodialysis. The position in the arterial part is preferred because the delay time is shorter. If positioned in the venous part a combination with a device for the discrimination of blood, gas or fluid as described by European Patent Application No. 0467805 is possible. Also, a combination with senors for other parameters is possible that can be measured optically such as urea or glucose absorbing in the infrared region.

The control of the ultrafiltration pump 250 by the control unit 260 as function of the signal of sensor 270 can be done as previously described. For choosing the appropriate control program and the control parameters, the control unit is equipped with appropriate input elements. Corresponding with the state of the art, microprocessors and screens can be used for the display and input components of the control unit 260.

We claim:

1. A method for controlling an ultrafiltration process for blood purification, which ultrafiltration process uses a central venous catheter for blood withdrawal, an extracorporeal circuit comprising a filter separated by a membrane into a blood side and a filtrate or dialysate side, a device for the removal of fluid from the filtrate or dialysate side which is controlled by a control unit, and at least one sensor for measuring the oxygen content in blood or filtrate or dialysate, said method comprising controlling the ultrafiltration as function of at least one parameter selected from the group oxygen concentration in blood or in filtrate or in dialysate.

2. The method of claim 1 in which oxygen concentration is measured by an oxygen sensor in the extracorporeal circuit selected from an oxygen saturation sensor or an oxygen partial pressure sensor.

3. The method of claim 1 in which oxygen concentration is measured by a sensor measuring the oxygen partial pressure in at least one of the spent dialysate and the filtrate.

4. The method of claim 1 in which a control unit for ultrafiltration control can be adjusted to stop ultrafiltration when the oxygen concentration decreases below an adjustable predetermined limit.

5. The method of claim 1 in which a control unit the ultrafiltration control controls the ultrafiltration rate as function of the deviation of a currently measured oxygen content from a starting value selected from a value at the beginning of the treatment and a maximum value measured during treatment.

6. The method of claim 5 comprising the additional step of stopping ultrafiltration at a predetermined lower limit for the oxygen content.

7. The method of claim 5 in which an ultrafiltration rate is controlled by a linear function connecting the value at the beginning of treatment and a lower limit.

8. The method of claim 1 in which the control unit employs fuzzy logic for ultrafiltration control as function of the oxygen content.

9. The method of claim 8 in which the fuzzy logic control uses the difference between the oxygen content at the beginning of dialysis and a value at the time of measurement, the difference between the actual value and an adjustable lower limit, and the rate of change.

10. The method of claim 1 in which at least one additional parameter in addition to the oxygen concentration parameter is used for ultrafiltration control.

11. The method of claim 10 in which the at least one additional parameter is selected from the group hematocrit, blood volume, blood pressure, blood temperature, body temperature, and the change of the plasma electrolyte concentration.

12. The method of claim 11 in which the dialysate temperature and the ultrafiltration rate are reduced when the oxygen content decreases simultaneously with an increase of the body temperature.

13. The method of claim 1 additionally comprising the step of using for blood return a conduit selected from the group central venous catheter and a cannula plus arteriovenous shunt.

14. The method of claim 1 additionally comprising the step of using a peripheral venous catheter for blood return.

15. The method of claim 1 additionally comprising the step of measuring oxygen concentration with an oxygen sensor between arterial blood access and blood pump.

16. The method of claim 1 additionally comprising the step of measuring oxygen concentration with an oxygen sensor between blood pump and blood treatment membrane device.

17. The method of claim 1 additionally comprising the step of measuring oxygen concentration with an oxygen sensor between the blood treatment membrane device and the venous drip or flow chamber.

18. The method of claim 1 additionally comprising the step of measuring oxygen concentration with an oxygen sensor between the venous drip or flow chamber and venous blood access.

19. The method of claim 1 additionally comprising the step of measuring oxygen concentration with an oxygen sensor in the blood part of the blood treatment membrane device.

20. The method of claim 1 in which the oxygen sensor can be used simultaneously for the discrimination between gas, liquid and blood.

21. An apparatus for ultrafiltration control during hemodialysis, hemofiltration, or hemodiafiltration with at least one central venous catheter, an extracorporeal circuit with a hemodialyzer, hemofilter or hemodiafilter, a dialysate or filtrate circuit, at least one sensor for measuring the oxygen content, and means for the controlled removal of fluid connected to a control unit, said apparatus comprising a control connection between the said oxygen sensor and the said control unit such that the ultrafiltration is controllable as a function of the oxygen content measured by said oxygen sensor.

22. The apparatus of claim 21 in which the oxygen content is measured by an oxygen sensor located in the extracorporeal circuit selected from the group oxygen saturation sensor and oxygen partial pressure sensor.

23. The apparatus of claim 21 in which the oxygen content is measured by an oxygen partial pressure sensor located in the dialysate or filtrate circuit.
